(19) United States
(12) Patent Application Publication
(10) Pub. No.: US 2019/0275191 A1
(43) Pub. Date: Sep. 12, 2019

(54) ANTI-MICROBIAL ARTICLES

(71) Applicant: 3M INNOVATIVE PROPERTIES COMPANY, St. Paul, MN (US)

(72) Inventors: Junkang Jacob Liu, Woodbury, MN (US); Ta-Hua Yu, Woodbury, MN (US); Amy K. McNulty, Stillwater, MN (US); Narina Y. Stepanova, Inver Grove Heights, MN (US); Badri Veeraraghavan, Woodbury, MN (US); Eric J. Olson, Waconia, MN (US); Matthew T. Scholz, Woodbury, MN (US); Ranjani V. Parthasarathy, Woodbury, MN (US); Kevin D. Hagen, St. Paul, MN (US)

(21) Appl. No.: 16/343,652

(22) PCT Filed: Oct. 5, 2017
(86) PCT No.: PCT/US2017/055363

§ 371 Date: Apr. 19, 2019

Related U.S. Application Data
(60) Provisional application No. 62/410,228, filed on Oct. 19, 2016.
(60) Provisional application No. 62/534,519, filed on Jul. 19, 2017.

(51) Int. Cl. A61L 15/18; A61L 15/44; A61L 15/46

(52) U.S. Cl. CPC A61L 15/18; A61L 15/44; A61L 2300/404; A61L 2300/104; A61L 2300/608; A61L 15/46

(57) ABSTRACT

An article having anti-microbial effect is provided. The article includes an occlusive layer; a substrate overlaying the occlusive layer, wherein the substrate having two opposing major surfaces; a metal oxide layer overlaying one opposing major surface of the substrate, wherein the metal oxide layer comprises a metal oxide; and a metal layer overlaying the other opposing major surface of the substrate; wherein the substrate is between the metal oxide layer and the metal layer; and wherein electric potential of the metal oxide layer is at least 0.454V more than electric potential of the metal layer.

### BACKGROUND

The risk of being infected from medical devices is particularly high in the medical field. Anti-microbial articles or coatings are used extensively to prevent/reduce infections in the medical community. For example, medical devices used by doctors, including orthopedic pins, plates and implants, wound dressings, etc., must constantly guard against infection. Metallic ions with anti-microbial properties, such as Ag, Au, Pt, Pd, Ir, Cu, Sn, Sb, Bi and Zn, were used as anti-microbial compounds. Of these metallic ions, silver is well known due to its highly effective bioactivity, and various silver salts, complexes and colloids have been greatly utilized in medical devices to prevent and control infection.

### SUMMARY

Although soluble salts of silver have been currently used to prevent microbial infections, they do not provide prolonged release of silver ions due to loss through removal or complexation of the free silver ions. They must be reapplied periodically to address this problem. Reapplication is often burdensome or even impractical, for example, when implanted medical devices are involved. Thus, it is desirable to have an anti-microbial article to provide a more effective and sustained release of anti-microbial agents.

In various exemplary embodiments described herein, the disclosed articles may be used to prevent microbial infections. The disclosed articles may be useful to provide an enhanced release of anti-microbial agents and thus to provide an enhanced anti-microbial activity.

In one aspect, the disclosure provides an article that includes an occlusive layer; a substrate overlaying the occlusive layer, wherein the substrate having two opposing major surfaces; a metal oxide layer overlaying one opposing major surface of the substrate, wherein the metal oxide layer comprises a metal oxide; and a metal layer overlaying the other opposing major surface of the substrate; wherein the substrate is between the metal oxide layer and the metal layer; and wherein electric potential of the metal oxide layer is at least 0.454V more than electric potential of the metal layer.

Other features and aspects of the present disclosure will become apparent by consideration of the detailed description.

### Definitions

Certain terms are used throughout the description and the claims that, while for the most part are well known, may require some explanation. It should be understood that, as used herein:

The terms “about” or “approximately” with reference to a numerical value or a shape means +/−five percent of the numerical value or property or characteristic, but also expressly includes any narrow range within the +/−five percent of the numerical value or property or characteristic as well as the exact numerical value. For example, a temperature of “about” 100° C. refers to a temperature from 95° C. to 105° C., but also expressly includes any narrower range of temperature or even a single temperature within that range, including, for example, a temperature of exactly 100° C.

The terms “a”, “an”, and “the” include plural referents unless the content clearly dictates otherwise. Thus, for example, reference to a material containing “a compound” includes a mixture of two or more compounds.

The term “or” is generally employed in its sense including “and/or” unless the content clearly dictates otherwise.

### BRIEF DESCRIPTION OF THE DRAWINGS

The disclosure may be more completely understood in consideration of the following detailed description of various embodiments of the disclosure in connection with the accompanying drawings, in which it is to be understood by one of ordinary skill in the art that the drawings illustrate certain exemplary embodiments only, and are not intended as limiting the broader aspects of the present disclosure.

FIG. 1 is a cross-sectional view of an embodiment of an anti-microbial article of the present disclosure.

### DETAILED DESCRIPTION

In the following description, reference is made to the accompanying set of drawings that form a part of the description hereof and in which are shown by way of illustration several specific embodiments. It is to be understood that other embodiments are contemplated and may be made without departing from the scope or spirit of the present invention. The following detailed description, therefore, is not to be taken in a limiting sense.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. At the very least, and not as an attempt to limit the application of the doctrine of equivalents to the scope of the claimed embodiments, each numerical parameter should at least be construed in light of the number of reported significant digits and by applying ordinary rounding techniques. In addition, the use of numerical ranges with endpoints includes all numbers within that range (e.g. 1 to 5 includes 1, 1.5, 2, 2.75, 3, 3.80, 4, and 5) and any narrower range or single value within that range.

Various exemplary embodiments of the disclosure will now be described with particular reference to the Drawings. Exemplary embodiments of the present disclosure may take on various modifications and alterations without departing from the spirit and scope of the disclosure. Accordingly, it is to be understood that the embodiments of the present disclosure are not to be limited to the following described exemplary embodiments, but are to be controlled by the limitations set forth in the claims and any equivalents thereof.

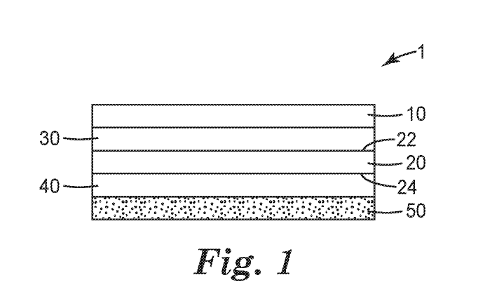

An article is disclosed herein. FIG. 1 is a cross-sectional view of an embodiment of article 1. Overall, article 1 includes an occlusive layer 10 and a substrate 20 overlaying the occlusive layer. The substrate has two opposing major surfaces 22 and 24. A metal oxide layer 30 overlays one opposing major surface of the substrate 20 and a metal layer 40 overlays the other opposing major surface of the substrate. In the embodiment shown in FIG. 1, metal oxide layer 30 overlays major surface 22 of the substrate 20 and metal layer 40 opposing major surface 24 of the substrate 20. Alternatively, metal oxide layer 30 may overlay major surface 24 of the substrate 20 and metal layer 40 may overlay major surface 22 of the substrate 20. In the embodiment shown in FIG. 1, metal oxide layer 30 adjoins substrate 20 and substrate 20 is next to metal layer 40. In some embodiments, can be in direct contact with one opposing major surface of the substrate and the metal layer can be in direct contact with the other opposing major surface of the substrate. In the embodiment shown in FIG. 1, the metal layer or metal oxide layer is continuous. Alternatively, the metal layer or metal oxide layer can be discontinuous. In some embodiments, the metal layer or metal oxide layer can be patterned.

The article of present disclosure may include additional layers between occlusive layer and the substrate, between the metal oxide layer and the substrate, between the metal layer and the substrate, or on the metal layer. For example, the article of present disclosure may include an additional adhesive layer between the metal layer and the substrate. In the embodiment shown in FIG. 1, an additional adhesive layer 50 can be supplied to article 1. In this embodiment, adhesive layer 50 covers the entire surface of metal layer 40. However, it is understood that the adhesive layer 50 may cover only a portion of the metal layer 40. The article may include an optional release liners (not shown) that covers all or a portion of the adhesives to prevent contamination of the adhesives. An optional carrier (not shown) may be included to cover all or a portion of occlusive layer 10, providing structural support if the article is thin and highly flexible. The carrier maybe removable from occlusive layer 10 once the article is placed on a subject. The article of present disclosure may include more than one substrate and adhesive layer (not shown).

The electric potential of the metal oxide layer may be different from the electric potential of the metal layer. In some embodiments, electric potential of the metal oxide layer is at least 0.454V, at least 1.240V, at least 1.557V, or at least 2.66V more than electric potential of the metal layer.

The article of the present disclosure can be used to provide an anti-microbial effect. The article can be provided to a health care provider and can be applied to a subject to release anti-microbial agents. The article of the present disclosure provide synergistic antimicrobial functionality, a faster contact kill performance with lower silver oxide coating, for example, less than 20 mg silver oxide per 100 cm², preferably less than 10 mg silver oxide per 100 cm² or even more preferably less than 5 mg silver oxide per 100 cm².

### Occlusive Layer

The occlusive layers are useful to provide an impermeable barrier to the passage of liquids and at least some gases. Representative barriers may include non-woven and woven fibrous webs, knits, films, foams polymeric films and other familiar backing materials. In some embodiments, a transparent occlusive layer is desirable to allow for viewing of the underlying subjects. Suitable occlusive layers may include those described in International Publication No. WO 2014/149718, the disclosures of which are hereby incorporated by reference.

In one embodiment, the occlusive layer has high moisture vapor permeability, but generally impermeable to liquid water so that microbes and other contaminants are sealed out from the area under the article. One example of a suitable material is a high moisture vapor permeable film such as described in U.S. Pat. Nos. 3,645,835 and 4,595,001, the disclosures of which are herein incorporated by reference. In one embodiment, the occlusive layer can be an elastomeric polyurethane, polyester, or polyether block amide films. These films combine the desirable properties of resiliency, elasticity, high moisture vapor permeability, and transparency. A description of this characteristic of occlusive layers can be found in issued U.S. Pat. Nos. 5,088,483 and 5,160,315, the disclosures of which are hereby incorporated by reference

Commercially available examples of potentially suitable materials for the occlusive layer may include the thin polymeric film sold under the trade names TEGADERM (3M Company), OPSITE (Smith & Nephew), etc. Because fluids may be actively removed from the sealed environments defined by the article, a relatively high moisture vapor permeable occlusive layer may not be required. As a result, some other potentially useful materials for the occlusive layer may include, e.g., metallocene polyolefins and SBS and SIS block copolymer materials could be used.

Regardless, however, it may be desirable that the occlusive layer be kept relatively thin to, e.g., improve conformability. For example, the occlusive layer may be formed of polymeric films with a thickness of 200 micrometers or less, or 100 micrometers or less, 50 micrometers or less, or 25 micrometers or less.

### Substrate

The substrate can be selected from foam, mesh, netting, woven, nonwoven, hydrocolloid, hydrogel, pressure sensitive adhesive and combination of thereof. In some embodiments, the substrate can be an absorbent substrate selected from foam, fabric, nonwoven, hydrocolloid, hydrogel or polymers with inherent microporosity, and combination of thereof. Exemplary absorbent substrate can include film, fabrics or porous article made from viscose, rayon, alginate, gauze, biopolymers, polyurethane, biodegradable polymers or the polymers described in U.S. Pat. No. 7,745,509, the disclosures of which is hereby incorporated by reference. The absorbent materials used in the absorbent substrate can be manufactured of any suitable materials including, but not limited to, woven or nonwoven cotton or rayon or netting and perforated film made from nylon, polyester or polyolefins. Absorbent pad can be used as the absorbent layer and can be useful for containing a number of substances, optionally including drugs for transdermal drug delivery, chemical indicators to monitor hormones or other substances in a patient, etc.

The absorbent layer may include a hydrocolloid composition, including the hydrocolloid compositions described in U.S. Pat. Nos. 5,622,711 and 5,633,010, the disclosures of which are hereby incorporated by reference. The hydrocolloid absorbent may comprise, for example, a natural hydrocolloid, such as pectin, gelatin, or carboxymethylcellulose (CMC) (Aqualon Corp., Wilmington, Del.), a semi-synthetic hydrocolloid, such as cross-linked carboxymethylcellulose (X4ink CMC) (e.g. Ac-Di-Sol; FMC Corp., Philadelphia, Pa.), a synthetic hydrocolloid, such as cross-linked polyacrylic acid (PAA) (e.g., CARBOPOL™ No. 974P; B.F. Goodrich, Brecksville, Ohio), or a combination thereof. Absorbent layer can be manufactured of other synthetic and natural hydrophilic materials including polymer gels and foams.

### Metal Oxide Layer

The metal oxide layer of the present disclosure includes a metal oxide. The metal oxide can be those known to have an anti-microbial effect. For most medical use, the metal oxide can also be biocompatible. In some embodiments, the metal oxide used in the metal oxide layer can include, but is not limited to, silver oxide, copper oxide, gold oxide, zinc oxide, magnesium oxide, titanium oxide, chromium oxide and combinations thereof. In some of these embodiments, the metal oxide can be silver oxide, including but not limited to, Ag₂O. In some embodiments, the metal oxide layer can include less than 50 wt. %, less than 40 wt. %, less than 20 wt. %, less than 10 wt. %, less than 5 wt. %, less than 1 wt. % non-oxidized metal. When the metal oxide layer includes more than 40 wt. % non-oxidized metal, the article will become more conductive, i.e., the resistivity of the article decreases, and the release of anti-microbial agents also decreases. In some embodiments, the article can include less than 40 mg, less than 20 mg or less than 5 mg silver oxide per 100 cm².

The metal oxide layer can be formed by any suitable means, for example, by physical vapor deposition techniques. The physical vapor deposition techniques can include, but is not limited to, vacuum or arc evaporation, sputtering, magnetron sputtering and ion plating. Suitable physical vapor deposition techniques can include those described in U.S. Pat. Nos. 4,364,995; 5,681,575 and 5,753,251, the disclosures of which are hereby incorporated by reference.

By the controlled introduction of reactive material, for example, oxygen into the metal vapor stream of vapor deposition apparatus during the vapor deposition of metals onto substrates, controlled conversion of the metal to metal oxides can be achieved. Therefore, by controlling the amount of the reactive vapor or gas introduced, the proportion of metal to metal oxide in the metal oxide layer can be controlled. For 100% conversion of the metal to metal oxides at a given level of the layer, at least a stoichiometric amount of the oxygen containing gas or vapor is introduced to a portion of the metal vapor stream. When the amount of the oxygen containing gas increases, the metal oxide layer will contain a higher weight percent of metal oxide. The ability to achieve release of metal atoms, ions, molecules or clusters on a sustainable basis can be effected by varying the amount of the oxygen containing gas. As the amount of metal oxide increases when the level of oxygen containing gas introduced increases, metal ions released from the article in turn increases. Thus, a higher weight percent of metal oxide can, for example, provide an enhanced release of anti-microbial agents, such as metal ions and provide an increased anti-microbial activity.

The metal oxide layer can be formed as a thin film. The film can have a thickness no greater than that needed to provide release of metal ions on a sustainable basis over a suitable period of time. In that respect, the thickness will vary with the particular metal in the coating (which varies the solubility and abrasion resistance), and with the amount of the oxygen containing gas or vapor introduced to the metal vapor stream. The thickness will be thin enough that the metal oxide layer does not interfere with the dimensional tolerances or flexibility of the article for its intended utility. Typically, the metal oxide layer has a thicknesses of less than 1 micron. However, it is understood that increased thicknesses may be used depending on the degree of metal ion release needed over a period of time.

The metal oxide layer can further comprise metal compounds such as silver chloride, silver bromide, silver iodide, silver fluoride, copper halide and zinc halide.

### Metal Layer

The metal layer of the present disclosure includes a metal. The metal can be those known to have a positive electric potential. In some embodiments, the metal oxide used in the metal oxide layer can include, but is not limited to, zinc, magnesium, aluminum, iron, calcium, tin, copper, titanium, chromium, nickel and alloys thereof. The metal oxide layer can be formed by any suitable means, for example, by vapor deposition techniques. The vapor deposition techniques can include, but is not limited to, vacuum or arc evaporation, sputtering, magnetron sputtering and ion plating. Suitable physical vapor deposition techniques can include those described in U.S. Pat. Nos. 4,364,995; 5,681,575 and 5,753,251, the disclosures of which are hereby incorporated by reference.

### Optional Components

Suitable polymer for use in the insulation coating layer can include polyethylene terephthlate, polystyrene, acrlonitrile butabiene styrene, polyvinyl chloride, polyvinylidene chloride, polycarbonate, polyacrylates, polyurethanes, polyvinyl acetate, polyvinyl alcohol, polyamide, polyimide, polypropylene, polyester, polyethylene, poly(methyl methacrylate), polyethylene naphthalate, styrene acrylonitrile copolymer, silicone-polyoxiamide polymers, fluoropolymers, cellulose triacetate polymer, cyclic olefin copolymers and thermoplastic elastomers. The insulation coating layer can be formed by any suitable means, including extrusion, solvent casting, or lamination process described in U.S. Pat. Nos. 3,415,920, 4,664,859 and 3,416,525.

Suitable adhesive for use in the article includes any adhesive that provides acceptable adhesion to skin and is acceptable for use on skin (e.g., the adhesive should preferably be non-irritating and non-sensitizing). Suitable adhesives are pressure sensitive and in certain embodiments have a relatively high moisture vapor transmission rate to allow for moisture evaporation. Suitable pressure sensitive adhesives include those based on acrylates, urethane, hyrdogels, hydrocolloids, block copolymers, silicones, rubber based adhesives (including natural rubber, polyisoprene, polyisobutylene, butyl rubber etc.) as well as combinations of these adhesives. The adhesive component may contain tackifiers, plasticizers, rheology modifiers as well as active components including for example an antimicrobial agent. Suitable adhesive can include those described in U.S. Pat. Nos. 3,389,827; 4,112,213; 4,310,509; 4,323,557; 4,595,001; 4,737,410; 6,994,904 and International Publication Nos. WO 2010/056541; WO 2010/056543 and WO 2014/149718, the disclosures of which are hereby incorporated by reference. The adhesive can be processed to form solid, pattern or porous adhesive layer.

Suitable release liners can be made of kraft papers, polyethylene, polypropylene, polyester or composites of any of these materials. In one embodiment, the package that contains the adhesive dressing may serve as a release liner. In one embodiment, the liners are coated with release agents such as fluorochemicals or silicones. For example, U.S. Pat. No. 4,472,480, the disclosure of which is hereby incorporated by reference, describes low surface energy perfluorochemical liners. In one embodiment, the liners are papers, polyolefin films, or polyester films coated with silicone release materials.

The carrier used in the article can be constructed of any suitable materials such as fabric that are woven or knitted, nonwoven material, papers, or film. In one embodiment, the carrier is along the perimeter of the occlusive layer and is removable from the occlusive layer, similar to the carrier used the 3M Tegaderm Transparent Film Dressing, available from 3M Company, St. Paul, Minn.

### Properties

The anti-microbial effect of the article can be achieved, for example, when the article is brought into contact with an alcohol or a water based electrolyte such as, a body fluid or body tissue, thus releasing metal ions such as Ag⁺, atoms, molecules or clusters. The concentration of the metal which is needed to produce an anti-microbial effect will vary from metal to metal. Generally, anti-microbial effect is achieved in body fluids such as plasma, serum or urine at concentrations less than 10 ppm. In some embodiments, Ag⁺ release concentration from the article can be 0.1 ppm, 0.5 ppm, 1 ppm, 2 ppm, 2.5 ppm, 3 ppm, 4 ppm, 5 ppm, 6 ppm, 7 ppm, 8 ppm, 9 ppm, 10 ppm, 20 ppm, 40 ppm or a range between and including any two of these values. As discussed above, when the amount of metal oxide in the metal oxide layer increases, the metal ions released from the article in turn increases. For example, a more than 60 wt. % metal oxide provides an enhanced release of metal ions from the article. Therefore, the article of the present disclosure can provide a very effective anti-microbial effect. In some embodiments, the article can exhibit a more than 4 log reduction of bacterial growth within 7 days.

The article can generate at least one electrical current when introduced to an electrolytic solution. In some embodiments, the article is capable of generating a current in a range from about 10 μA to about 5000 μA when introduced to an electrolytic solution. In some embodiments, the article is capable of generating a current in a range from about 100 μA to about 1000 μA when introduced to an electrolytic solution. In the presence of an electrically conducting solution, redox reactions may take place, and thus currents may be produced between the metal oxide layer and the metal layer. For example, when the metal oxide layer includes silver oxide and the metal layer includes zinc, silver oxide is the cathode (positive electrode) and zinc is the anode (negative electrode), because the electrons follow from zinc to silver oxide.

The flow of ions generates the electrical current. Thus, when the article of present application is used as a wound dressing, it can recreate a physiologic current, which is important to the induction of neutrophil, macrophage and fibroblast cells essential to the healing process. In addition, the current can stimulates regionalnerve endings to promote their involvement in wound resolution. Further, the current can inhibit the growth of bacteria. Therefore, the current generated by the article can have synergistic antimicrobial functionality along with Ag⁺ release from the article.

The article can generate a more than 6.2, more than 6.5, more than 7, more than 8, more than 9 pH of when in contact with water. Not bound by the theory, the higher pH level generated when the article is in contact with water, may enhance the antimicrobial functionality of the article.

Various exemplary embodiments of the present disclosure are further illustrated by the following listing of embodiments, which should not be construed to unduly limit the present disclosure:

### EMBODIMENTS

Embodiment 1 is an article comprising: an occlusive layer; a substrate overlaying the occlusive layer, wherein the substrate having two opposing major surfaces; a metal oxide layer overlaying one opposing major surface of the substrate, wherein the metal oxide layer comprises a metal oxide; and a metal layer overlaying the other opposing major surface of the substrate; wherein the substrate is between the metal oxide layer and the metal layer; and wherein electric potential of the metal oxide layer is at least 0.454V more than electric potential of the metal layer.

Embodiment 2 is the article of embodiment 1, wherein the electric potential of the metal oxide layer is at least 1.240V more than the electric potential of the metal layer.

Embodiment 3 is the article of any of embodiments 1 to 2, wherein the electric potential of the metal oxide layer is at least 1.557V more than the electric potential of the metal layer.

Embodiment 4 is the article of any of embodiments 1 to 3, wherein the electric potential of the metal oxide layer is at least 2.66V more than the electric potential of the metal layer.

Embodiment 5 is the article of any of embodiments 1 to 4, wherein the metal oxide layer comprises less than 50 wt. % non-oxidized metal.

Embodiment 6 is the article of any of embodiments 1 to 5, wherein the metal oxide layer comprises less than 40 wt. % non-oxidized metal.

Embodiment 7 is the article of any of embodiments 1 to 6, wherein the article is capable of generating a more than 6.5 pH of when in contact with water.

Embodiment 8 is the article of any of embodiments 1 to 7, wherein the article is capable of generating a current in a range from about 10 μA to about 5000 μA when introduced to an electrolytic solution.

Embodiment 9 is the article of any of embodiments 1 to 8, wherein the article is capable of generating a current in a range from about 100 μA to about 1000 μA when introduced to an electrolytic solution.

Embodiment 10 is the article of any of embodiments 1 to 9, wherein the metal oxide layer or the metal layer is discontinuous or patterned.

Embodiment 11 is the article of any of embodiments 1 to 10, wherein the metal oxide layer is in direct contact with one opposing major surface of the substrate and the metal layer is in direct contact with the other opposing major surface of the substrate.

Embodiment 12 is the article of any of embodiments 1 to 11, wherein the substrate is selected from foam, mesh, netting, woven, nonwoven, cotton, cellulose fabrics, perforated film, hydrocolloid, hydrogel, polymers with inherent porosity, pressure sensitive adhesive and combination of thereof.

Embodiment 13 is the article of any of embodiments 1 to 12, wherein the metal oxide is selected from silver oxide, copper oxide, gold oxide, platinum oxide, zinc oxide, magnesium oxide, titanium oxide, chromium oxide and combinations thereof.

Embodiment 14 is the article of any of embodiments 1 to 13, wherein the metal oxide is silver oxide.

Embodiment 15 is the article of embodiment 14, wherein the silver oxide is Ag₂O.

Embodiment 16 is the article of any of embodiments 1 to 15, wherein the metal layer comprises a metal and the metal is selected from zinc, magnesium, aluminum, iron, calcium, tin, copper, titanium, chromium, nickel and alloys thereof.

Embodiment 17 is the article of any of embodiments 1 to 16, wherein Ag⁺ release concentration of the article is more than 0.1 ppm.

Embodiment 18 is the article of any of embodiments 1 to 17, wherein the article comprises less than 40 mg silver oxide per 100 cm².

Embodiment 19 is the article of any of embodiments 1 to 18, wherein the article comprises less than 20 mg silver oxide per 100 cm².

Embodiment 20 is the article of any of embodiments 1 to 19, wherein the article comprises less than 5 mg silver oxide per 100 cm².

### EXAMPLES

These Examples are merely for illustrative purposes and are not meant to be overly limiting on the scope of the appended claims. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements. At the very least, and not as an attempt to limit the application of the doctrine of equivalents to the scope of the claims, each numerical parameter should at least be construed in light of the number of reported significant digits and by applying ordinary rounding techniques.

### Summary of Materials

Unless otherwise noted, all parts, percentages, ratios, etc. in the Examples and the rest of the specification are by weight. Solvents and other reagents used may be obtained from Sigma-Aldrich Chemical Company (Milwaukee, Wis.) unless otherwise noted. In addition, Table 1 provides abbreviations and a source for all materials used in the Examples below:

TABLE 1
Materials and sources.

| | Tradename or | |
|---|---|---|
| Material | Abbreviation | Source (Location) |
| Silver sputtering target, | None | |
| 76.2 mm diameter | | |
| Copper sputtering target, | None | |
| 76.2 mm diameter | | |
| Magnesium sputtering target, | None | |
| 76.2 mm diameter | | |
| Zinc sputtering target, | None | |
| 76.2 mm diameter | | |
| Viscose fabric | WC135 | Fibertex Nonwovens |
| Silver-coated antimicrobial | Acticoat Surgical Foam | Smith & Nephew (Andover, |
| dressing | | MA) |
| Phosphate buffer | 3M Butterfield's buffer | 3M Company (St. Paul, MN) |
| Nutrient broth | Difco Tryptic Soy Broth | VWR (Radnor, PA) |
| Maximum Recovery Diluent | MRD | |
| Fetal Bovin Serum | FBS | |
| D/E neutralizing broth | Difco D/E neutralizing broth | BD (Franklin Lakes, NJ) |
| 3M Petrifilm Plates | 3M Petrifilm | 3M Company (St. Paul, MN) |
| Potato Dextrose Agar plates | PDA BBL | VWR (Radnor, PA) |

### Methods

### Sputtering Deposition Process

Silver films were coated onto 152 mm by 152 mm substrates by magnetron physical vapor deposition. The films were sputtered from a 76.2 mm diameter round silver target in a batch coater. The substrate was placed on a substrate holder set up inside a vacuum chamber with a sputtering metal target located at a height of 228.6 mm above the substrate holder. After the chamber was evacuated to 2×10⁻⁵ torr base pressure, sputter gases of argon (71% by flow rate) and reactive oxygen (29% by flow rate) were admitted inside the chamber and total pressure of the chamber was adjusted to 5 millitorr. Sputtering was initiated using a DC power supply at a constant power level of 0.25 kilowatts. The sputtering duration was varied to produce a coating weight per unit area of 0.05 mg/cm².

Copper films were sputtered from a 76.2 mm diameter round copper target in a batch coater. The substrate was placed on a substrate holder set up inside a vacuum chamber with a sputtering metal target located at a height of 228.6 mm above the substrate holder. After the chamber was evacuated to 2×10⁻⁵ torr base pressure, argon was admitted inside the chamber and total pressure of the chamber was adjusted to 1.6 millitorr. Sputtering was initiated using a DC power supply at a constant power level of 0.50 kilowatts for 5 minutes and 30 seconds.

Magnesium films were sputtered from a 76.2 mm diameter round magnesium target in a batch coater. The substrate was placed on a substrate holder set up inside a vacuum chamber with a sputtering metal target located at a height of 228.6 mm above the substrate holder. After the chamber was evacuated to 2×10⁻⁵ torr base pressure, argon was admitted inside the chamber and total pressure of the chamber was adjusted to 1.6 millitorr. Sputtering was initiated using a DC power supply at a constant power level of 0.50 kilowatts for 15 minutes.

Zinc films were sputtered from a 76.2 mm round zinc target in a batch coater. The substrate was placed on a substrate holder set up inside a vacuum chamber with a sputtering metal target located at a height of 228.6 mm above the substrate holder. After the chamber was evacuated to 2×10⁻⁵ torr base pressure, argon was admitted inside the chamber and total pressure of the chamber was adjusted to 1.6 millitorr. Sputtering was initiated using a DC power supply at a constant power level of 0.50 kilowatts for 1.5 minutes.

### Cosputtering Deposition Process

Cosputtering of silver and copper was performed using a PVD 75 batch coater (Kurt J. Lesker Company, Jefferson Hills, Pa.) by magnetron physical vapor deposition. The substrate was placed on a substrate holder set up inside a vacuum chamber. The substrate holder was located at a distance of 228.6 mm above the sputtering metal targets. After the chamber was evacuated to 9×10⁻⁶ torr base pressure, sputtering gases of argon (70% by flow rate) and reactive oxygen (30% by flow rate) were admitted inside the chamber and the total pressure of the chamber was adjusted to 6 millitorr. Sputtering was initiated using a DC power supply at a constant power level of 0.05 kilowatts to a 76.2 mm silver target and a RF power supply at a power level of 0.2 kilowatts to a 76.2 mm copper target. The sputtering duration was selected to produce a coating weight per unit area of 0.05 mg/cm².

### Antibacterial Log Reduction Testing Method

This contact test method was used to evaluate the antibacterial activity of the coatings in the presence of artificial would fluid. The bacterial inoculum of Staphylococcus aureus ATCC 6538 was prepared in artificial would fluid. Artificial Would Fluid was prepared by mixing Maximum Recovery Diluent and Fetal Bovime Serum in 1:1 ratio. The sample was pre-saturated with artificial wound fluid and then a portion of the bacterial suspension (250 microliters) was placed onto the surface of the article and the inoculated article was incubated for the specified contact time at 37+/−1° C. After incubation, the article was placed into 20 ml of D/E Neutralizing Broth. The number of surviving bacteria in the Neutralizing broth was determined by plating serial dilutions using 3M Petrifilm.

### Antifungal Log Reduction Testing Method

This contact test method was used to evaluate the antifungal activity of the coatings without the presence of artificial would fluid. The Candida albicans ATCC 10231 (yeast) inoculum was prepared in a solution of 1 part Nutrient Broth (NB) and 499 parts phosphate buffer. A portion of the fungal suspension (150 ul) was placed onto the surface of the sample and the inoculated sample was incubated for the specified contact time at 27+/−1° C. After incubation, the sample was placed into 20 ml of D/E Neutralizing Broth. The number of surviving yeast in the Neutralizing broth was determined by plating serial dilutions using Potato Dextrose Agar.

### Examples 1-3 and Comparative Examples 1-5

For Examples 1-3, silver oxide (AgOx) was deposited on one side of viscose and a second metal of 75 nm thickness (Mg, Zn or Cu) was deposited on the second side, as indicated in Table 2. For Comparative Examples 1-3, no AgOx was deposited on viscose, but a second metal was deposited, corresponding to the second metal deposited on Examples 1-3. Comparative Example 4 was fabricated by depositing AgOx at the higher level of 40 mg/100 cm², but no second metal was deposited. Commercially available Acticoat Surgical Foam was used as Comparative Example 5. The contact kill performance of all Examples and Comparative Examples was tested in artificial wound fluid as described in Antibacterial Log Reduction Testing Method. Significantly improved kill performance was observed in Example 1 compared to Comparative Example 1, in Example 2 compared to Comparative Example 2, and in Example 3 compared to Comparative Example 3. Examples 1-3 also performed well in comparison to Comparative Examples 4 and 5, which had much higher silver loadings than the Examples. These bimetal articles clearly demonstrate synergistic antimicrobial efficacy for faster kill performance at much lower Ag coating weight in contrast to their comparative examples.

TABLE 2
Construction and kill performance of Examples 1-3 (EX) and Comparative Examples 1-5 (CE).

| | | | Second Metal | | Log reduction in artificial wound fluid | |
|---|---|---|---|---|---|---|
| Sample | Substrate | Silver content | (thickness) | | 2 hour exposure | 24 hour exposure |
| CE 1 | Viscose | None | Mg | (75 nm) | None | None |
| EX 1 | Viscose | AgOx, 5 mg/100 cm² | Mg | (75 nm) | 2 | >6 |
| CE 2 | Viscose | None | Zn | (75 nm) | 0.3 | >6 |
| EX 2 | Viscose | AgOx, 5 mg/100 cm² | Zn | (75 nm) | 1 | >6 |
| CE 3 | Viscose | None | Cu | (75 nm) | None | 1.72 |
| EX 3 | Viscose | AgOx, 5 mg/100 cm² | Cu | (75 nm) | 0.45 | >6 |

| | | | | | |
|---|---|---|---|---|---|
| CE 4 | Viscose | AgOx, 40 mg/100 | None | 0.59 | >6 |
| | | cm² | | | |
| CE 5 | Acticoat | AgOx, 90 mg/100 | None | 0.45 | >6 |
| | Surgical Foam | cm² | | | |

### Example 4

Mixture of silver and copper was co-deposited on one side of viscose and Mg was deposited on the second side of viscose. The antifungal log reduction in aqueous solution after 24 hour exposure against Candida albicans (fungus) was 3.5.

All references and publications cited herein are expressly incorporated herein by reference in their entirety into this disclosure. Illustrative embodiments of this invention are discussed and reference has been made to possible variations within the scope of this invention. For example, features depicted in connection with one illustrative embodiment may be used in connection with other embodiments of the invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. An article comprising:
an occlusive layer;
a substrate overlaying the occlusive layer, wherein the substrate having two opposing major surfaces;
a metal oxide layer overlaying one opposing major surface of the substrate, wherein the metal oxide layer comprises a metal oxide; and
a metal layer overlaying the other opposing major surface of the substrate;
wherein the substrate is between the metal oxide layer and the metal layer; and
wherein electric potential of the metal oxide layer is at least 0.454V more than electric potential of the metal layer.

2. The article of claim 1, wherein the electric potential of the metal oxide layer is at least 1.240V more than the electric potential of the metal layer.

3. The article of claim 1, wherein the electric potential of the metal oxide layer is at least 1.557V more than the electric potential of the metal layer.

4. The article of claim 1, wherein the electric potential of the metal oxide layer is at least 2.66V more than the electric potential of the metal layer.

5. The article of claim 1, wherein the metal oxide layer comprises less than 50 wt. % non-oxidized metal.

6. The article of claim 1, wherein the metal oxide layer comprises less than 40 wt. % non-oxidized metal.

7. The article of claim 1, wherein the article is capable of generating a more than 6.5 pH of when in contact with water.

8. The article of claim 1, wherein the article is capable of generating a current in a range from about 10 μA to about 5000 μA when introduced to an electrolytic solution.

9. The article of claim 1, wherein the article is capable of generating a current in a range from about 100 μA to about 1000 μA when introduced to an electrolytic solution.

10. The article of claim 1, wherein the metal oxide layer or the metal layer is discontinuous or patterned.

11. The article of claim 1, wherein the metal oxide layer is in direct contact with one opposing major surface of the substrate and the metal layer is in direct contact with the other opposing major surface of the substrate.

12. The article of claim 1, wherein the substrate is selected from foam, mesh, netting, woven, nonwoven, cotton, cellulose fabrics, perforated film, hydrocolloid, hydrogel, polymers with inherent porosity, pressure sensitive adhesive and combination of thereof.

13. The article of claim 1, wherein the metal oxide is selected from silver oxide, copper oxide, gold oxide, platinum oxide, zinc oxide, magnesium oxide, titanium oxide, chromium oxide and combinations thereof.

14. The article of claim 1, wherein the metal oxide is silver oxide.

15. The article of claim 14, wherein the silver oxide is Ag₂O.

16. The article of claim 1, wherein the metal layer comprises a metal and the metal is selected from zinc, magnesium, aluminum, iron, calcium, tin, copper, titanium, chromium, nickel and alloys thereof.

17. The article of claim 1, wherein Ag⁺ release concentration of the article is more than 0.1 ppm.

18. The article of claim 1, wherein the article comprises less than 40 mg silver oxide per 100 cm².

19. The article of claim 1, wherein the article comprises less than 20 mg silver oxide per 100 cm².

20. The article of claim 1, wherein the article comprises less than 5 mg silver oxide per 100 cm².
